(19) United States
(12) Patent Application Publication
(10) Pub. No.: US 2010/0069716 A1
(43) Pub. Date: Mar. 18, 2010

(54) FLEXIBLE GUIDE CONDUIT

(76) Inventors: Yem CHIN, Burlington, MA (US); Paul DiCARLO, Middleboro, MA (US); Kurt GEITZ, Sudbury, MA (US); Jon T. McINTYRE, Newton, MA (US); Stephen J. PERRY, Shirley, MA (US)

Correspondence Address: FAY KAPLUN & MARCIN, LLP, 150 BROADWAY, SUITE 702, NEW YORK, NY, 10038, US

(21) Appl. No.: 12/548,872

(22) Filed: Aug. 27, 2009

Related U.S. Application Data
(60) Provisional application No. 61/096,519, filed on Sep. 12, 2008.

(51) Int. Cl. A61B 1/00

(52) U.S. Cl. 600/114

(57) ABSTRACT

A guide conduit for facilitating insertion of flexible instruments into body lumens, the conduit defining a lumen extending from an opening at a proximal end of the conduit to a distal opening at the distal end of the conduit, the conduit comprises an outer layer forming a substantially smooth outer surface and a plastically deformable layer radially within the outer layer, the plastically deformable inner layer constructed to maintain its shape when subjected to a force below a predetermined threshold level and to assume a new shape when subjected to a bending force greater than the threshold level, wherein the threshold level is selected to be greater than a range of forces to which the conduit will be subjected by instruments inserted therethrough.

### PRIORITY CLAIM

This application claims priority to U.S. Provisional Application Ser. No. 61/096,519 entitled “Flexible Guide Conduit” filed Sep. 12, 2008, The specification of the above-identified application is incorporated herewith by reference.

### FIELD OF INVENTION

The present invention relates to devices facilitating the insertion of instruments through body lumens.

### BACKGROUND

Many procedures require the insertion of a flexible instrument (e.g., an endoscope) into a body lumen. During these procedures, a diagnostician may navigate the lumen using a steerable endoscopic tip or, alternatively, by performing a series of torquing, pushing and pulling maneuvers of the proximal end of the device to advance and direct the distal end. The forces applied to the instrument are transferred to the surrounding tissue and may be problematic and painful. Movement of the instrument may be impeded by frictional engagement with the walls of the lumen making it difficult to advance or withdraw the instrument, in some cases preventing the instrument from reaching a target area. In addition, the stress applied to the lumen may generate painful spasms or perforate the lumen. These factors extend the time, discomfort and risk associated with these procedures.

Guides have been developed to absorb this stress and facilitate insertion of the endoscope while minimizing the impact on the lumenal walls. However, current guides are often expensive, bulky and/or require added steps for insertion and retraction, which unduly complicate the procedures.

### SUMMARY OF THE INVENTION

The present invention is directed to a guide conduit for facilitating insertion of flexible instruments into body lumens, the conduit defining a lumen extending from an opening at a proximal end of the conduit to a distal opening at the distal end of the conduit, the conduit comprising an outer layer forming a substantially smooth outer surface and a plastically deformable layer radially within the outer layer, the plastically deformable inner layer constructed to maintain its shape when subjected to a force below a predetermined threshold level and to assume a new shape when subjected to a bending force greater than the threshold level, wherein the threshold level is selected to be greater than a range of forces to which the conduit will be subjected by instruments inserted therethrough.

### BRIEF DESCRIPTION OF DRAWINGS

FIG. 1 is a side view of a first embodiment of a flexible conduit according to the invention;

FIG. 2 is a sectional side view of a distal end of a first embodiment of the flexible conduit according to the invention;

FIG. 3 is a sectional side view of a distal end of a first embodiment of the flexible conduit according to the invention;

FIG. 4 is a detail view of the first embodiment of a flexible conduit according to the invention;

FIG. 5 is a perspective view of the employment of a first embodiment of the flexible conduit according to the invention;

FIG. 6 is a second perspective view of the employment of a first embodiment of the flexible conduit according to the invention;

FIG. 7 is a graph of the force and torque applied to a colonoscope during a typical procedure;

FIG. 8 is a graph of the hypothesized measurement of the force and torque applied to a colonoscope under employment of the present invention;

FIG. 9 is a perspective view showing forces applied to an element of a flexible conduit to lock the conduit, according to the invention;

FIG. 10 is a graph of the tension forces applied to the present invention;

FIG. 11 is a partial side view of another embodiment of the present invention;

FIG. 12 is a partial side view of another embodiment of the present invention

FIG. 13A is a partial side view of another embodiment of the present invention;

FIG. 13B is a perspective view of another embodiment of the present invention;

FIG. 14 is a partial side view of another embodiment of the present invention;

FIGS. 15-17 are details views showing the element of FIG. 14;

FIG. 18 is a partial side view of another embodiment of the present invention;

FIG. 19 is a detail view showing the element of FIG. 18;

FIG. 20 is a partial side view of another embodiment of the present invention;

FIG. 21 is a detail view showing the element of FIG. 20;

FIG. 22 is a partial side view of another embodiment of the present invention;

FIG. 23 is a partial side view of another embodiment of the present invention;

FIG. 24 is a perspective view of another embodiment of the present invention;

FIG. 25 is a partial side view of another embodiment of the present invention;

FIG. 26 is a partial side view of another embodiment of the present invention;

FIG. 27 is a side view of another embodiment of the present invention;

FIG. 28 is a side view of another embodiment of the present invention; and

FIG. 29 is a side view of another embodiment of the present invention.

### DETAILED DESCRIPTION

The present invention may be further understood with reference to the following description and appended drawings, wherein like elements are referred to with the same reference numerals. The present invention relates to guides for the insertion of flexible endoscopes or other flexible elongate instruments along tortuous body lumen paths. The exemplary embodiments are described herein in conjunction with flexible endoscopes. However, those skilled in the art will understand that the guides may be used to facilitate the insertion of any flexible instrument through a body lumen and that the reference to the use of endoscopes is exemplary and not intended to limit the invention.

Embodiments of the flexible instrument guide according to the present invention may be used in procedures such as, but not limited to, colonoscopy, enteroscopy, exploration of the biliary tree, exploration of the GI tract and of extra-lumenal space, among others. These methods and devices are useful for screening and diagnostic purposes, as well as for a host of treatments. Alternatively, guides according to the present invention may also be used surgical procedures requiring surgical access in the body such as for examples, natural orifice transluminal endoscopic surgery (“NOTES”) or any procedure requiring incisions on the body.

A guide and a method of use of the guide are described in which the guide is sufficiently longitudinally flexible to be inserted along a path defined by an endoscope but which is longitudinally plastically deformed as it slides along the endoscope to relatively rigidly assume the shape of the endoscope. Then, as additional length of the endoscope is inserted through the guide, the stresses exerted outward from the endoscope along the turns are absorbed by the guide and are not transferred to surrounding tissue. The longitudinal rigidity of guides according to certain embodiments of the invention may be varied during the procedure so that the guide may be inserted and removed while in a more flexible state and rigidized when it has assumed a desired shape. The guides according to the invention are also preferably torsionally rigid to facilitate the placement of distal ends thereof at desired locations and/or in desired rotational orientations. A working lumen extending through the guides is sized to allow passage therethrough of a flexible instrument to be used therewith.

A guide according to a first embodiment of the invention comprises a conduit facilitating the movement of a flexible instrument such as an endoscope through a body lumen. The conduit is preferably plastically deformable so that its shape may be conformed to the tortuous path of a body lumen yet, once placed in such a conforming shape, rigid enough to absorb a substantial portion of the forces applied by a flexible instrument inserted therethrough to minimize the transmission of these forces to the walls of the lumen. The conduit may, according to certain embodiments, be formed of a plurality of elements coupled so that they may be moved relative to one another by the application of forces thereto above a predetermined threshold while resisting relative movement when subjected to forces below the threshold.

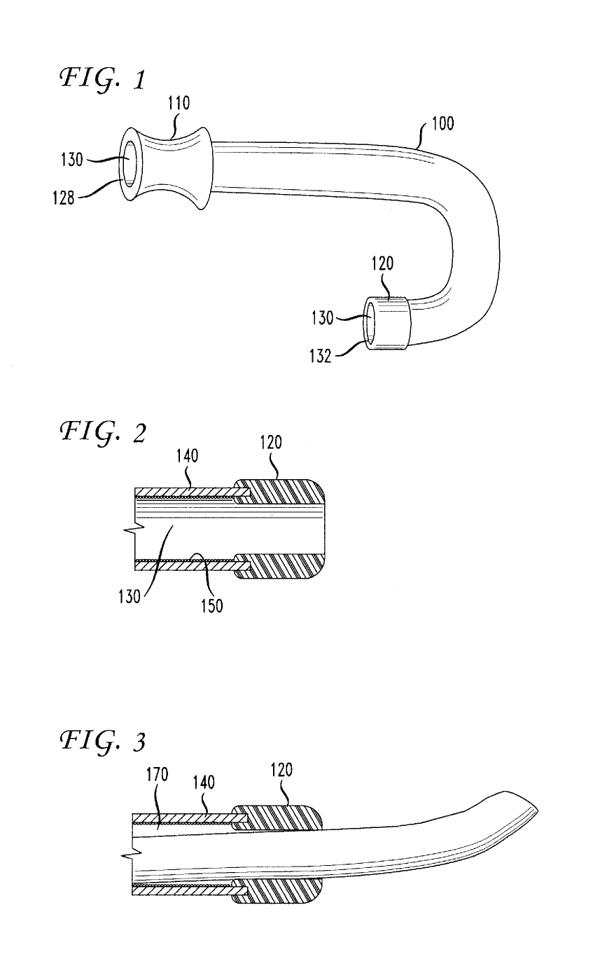

As shown in FIG. 1, a guide according to an exemplary embodiment of the present invention includes a substantially tubular body 100 extending from a proximal handle 110 to a distal tip 120. The handle 110 may be ergonomically designed to facilitate grasping and manipulation of the substantially tubular body 100 for insertion of the substantially tubular body 100 into a body lumen. The handle 110 may include a proximal opening 128 providing access to a lumen 130 extending through the substantially tubular body 100 to a distal opening 132 formed in the distal end 120, as further detailed in FIGS. 2 and 3. The proximal opening 128 permits the introduction of a flexible instrument such as an endoscope into the lumen 130 so that the instrument may be advanced through the lumen 130 to the distal opening 132. The substantially tubular body 100 according to this embodiment may also comprise a coil 150 wound coaxially about an axis of the substantially tubular body 100 along an inner surface of the lumen 130. The coil 150 is adapted to reinforce the inner diameter of the lumen 130 against radially inwardly directed external forces applied to the substantially tubular body 100 during insertion into and movement through the lumen.

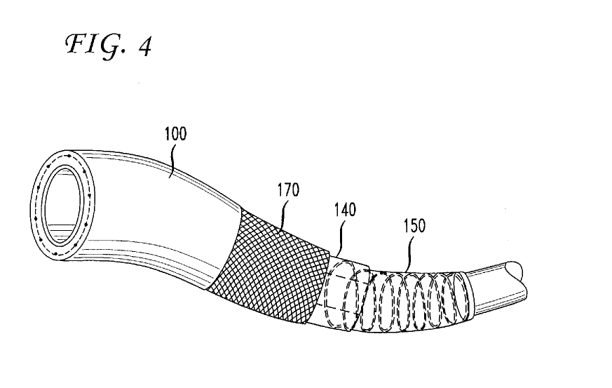

The substantially tubular body 100 may also contain one or more wires 140 running parallel to or in a helical fashion about an axis of the tube. The wires 140 may, for example, be distributed at regular intervals around the circumference of the substantially tubular body 100. The wires 140 may provide further structural support to the substantially tubular body 100. The substantially tubular body may also contain a tubular braid 170 of intertwined metal or plastic wires substantially coaxial with the substantially tubular body 100 and adapted to transfer torque along the axis of the substantially tubular body 100. The coil 150, the wires 140 and the tubular braid 170 may be layered over one another along the length of the inner wall of the lumen 130, as shown in FIG. 4.

The distal tip 120 may be tapered to provide a smooth transition from the outer surface of the substantially tubular body 100 to the outer surface of a flexible instrument inserted therethrough to minimize trauma to lumenal tissue. In addition, an inner diameter of the tip 120 is selected to closely fit an outer diameter of the flexible instrument to be inserted therethrough to prevent the capture of mucosa and/or the leakage of air, gas or fluids into the lumen 130 of the substantially tubular body 100.

An inner surface of the lumen 130 of the substantially tubular body 100 and/or an inner surface of the distal tip 120 may be treated to reduce friction, for example, through the application of a hydrophilic coating which, when wetted, provides a lubricious interface with the flexible instrument inserted into the lumen 130. Alternatively, friction in the inner surface of the lumen 130 may also be reduced by employing a series of one of longitudinal and circumferentially formed ridges to minimize a contacting surface area of the lumen 130, as those skilled in the art will understand.

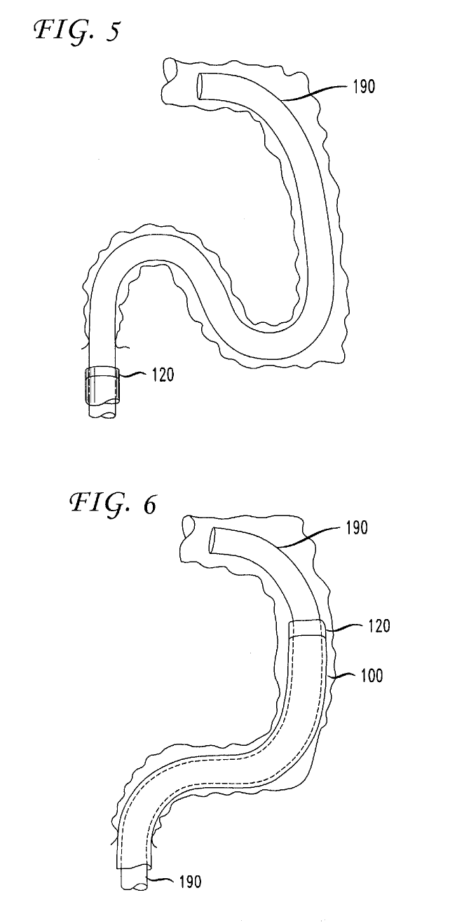

As shown in FIGS. 5 and 6, once an endoscope 190 has been inserted by a distance into a body lumen, the substantially tubular body 100 may be advanced over the endoscope 190 and into the body lumen. This method may be repeated successively, advancing the endoscope 190 and the substantially tubular body 100 in increments until the desired treatment area has been reached, as illustrated in FIG. 6.

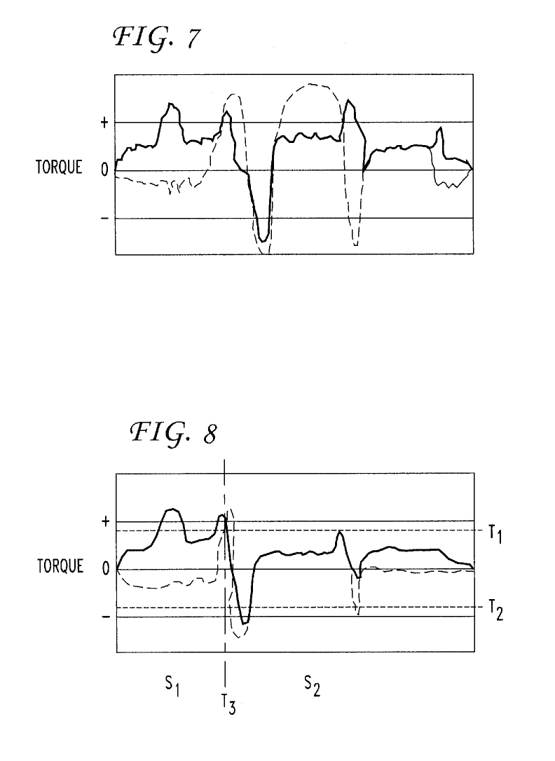

FIG. 7 portrays a typical measure of the input pattern and torque applied to a colonoscope during a procedure, wherein it is evident that an excessive amount of torque is being applied to the colon. Employment of the substantially tubular body 100 of the present invention reduces the relative input force and push/pull torque exerted on the colonoscope, as shown in FIG. 8. The force/torque needed to reshape the guide is indicated by the phantom lines T₁ and T₂. The phantom line T₃ separates regions S₁ and S₂ of the graph which correspond to torque being applied to the colon before and after employment of the guide of the present invention, respectively. The force/torque needed to reshape the guide of the present invention is indicated by the phantom lines T₁ and T₂, wherein it is evident that the guide of the present invention applies a low torque to the colon, thereby minimizing and/or preventing discomfort and trauma to the patient

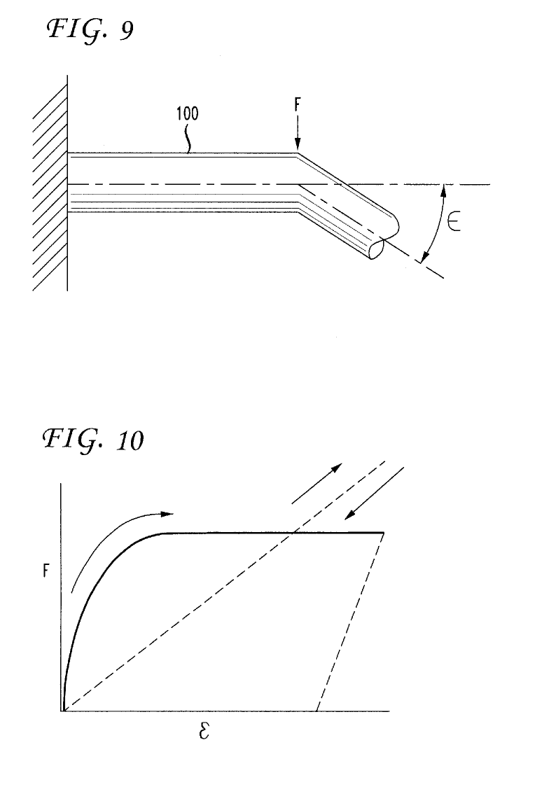

The material of the substantially tubular body 100 of the present invention may exhibit a stiffness yielding substantially the properties illustrated in FIG. 9. Those skilled in the art will understand that the stiffness of the guide may be varied in accordance with the requirements of the procedure in question. For example, a lumen exhibiting tight curves generally requires a material more flexible than that suitable for lumens with less curves around larger radii. In fabrication and testing of the stiffness of the material of the substantially tubular body 100, a deflection angle ε is defined in accordance with an external force F applied to the substantially tubular body 100 during the procedure. As shown in FIG. 9, the substantially tubular body 100 of the present invention deflect by the angle ε in response to a force F exerted thereagainst in any direction. As illustrated in FIG. 10 the deflection angle e may then be used to calculate a maximum load bearable by the material based on a materials property curve, as those skilled in the art will understand.

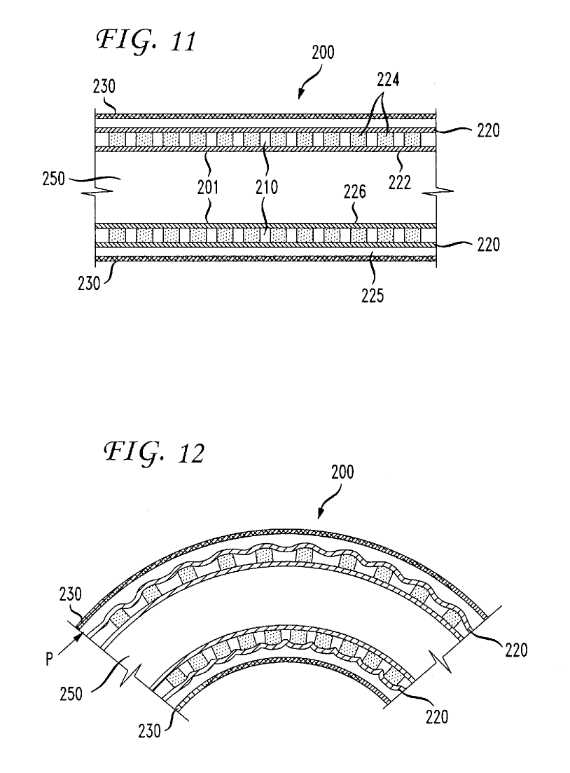

As illustrated in FIG. 11, an alternate embodiment of the present invention may include a guide sheath 200 comprising a slotted member 201 placed circumferentially within a pair of tubes 220, 230, respectively, sealed to one another at proximal and distal ends to form an annular space 225 that may be inflated. The inside wall 226 of the sheath 200 may be formed by lining an inner surface of the slotted member 201 with a low friction material (e.g., a polymer, Teflon, etc.) to further facilitate movement of the endoscope through a lumen 250 extending through the sheath 200. The two tubes 220, 230, enveloping the slotted member 201 can enhance the column strength of the sheath 200 while the central coupling 222 joining adjacent ones of the ribs 224 to each other can provide for the transmission of torque along the length of the sheath 200 while the outer tube 230 and the inside wall 226 provide smooth outer surfaces to reduce trauma to lumenal tissue and/or to endoscopes or other devices passed through the lumen 250. The inner tube 220, situated between the slotted member 201 and the outer tube 230, may be preferably formed to be more compliant than the outer tube 230. As would be understood by those skilled in the art, this may be accomplished by any of various methods including, for example, forming the outer tube 230 of a thicker layer of the same material comprising the inner tube 220, forming the inner tube 220 of a material more compliant than that of the outer tube 230 or a combination of these methods. An inflation port (not shown) in fluid communication with the annular space 225 may be formed near a proximal end of the sheath 200 for connection to a source of fluid to allow pressurization of the annular space 225. Thus, while both the inner and outer tubes 220, 230, respectively, are both formed of elastomeric, compliant materials to provide required flexibility for the sheath 200, the outer tube 230 can be substantially more resistant to deformation than the innermost tube layer 220.

The slots 210 are adapted to enable the slotted member 201 to bend around and conform to the curves of body lumens into which the sheath 200 is inserted. The slots 210 can assume any configuration including, but not limited to, holes. Specifically, when the sheath 200 is inserted past a curve in a body lumen, the slots 210 on a side of the slotted member 201 facing a radially outer part of the curve spread apart from one another creating gaps of increased width between the adjacent slots 210 while the slots 210 on a side of the slotted member 201 facing a radially inner part of the curve, a pushed toward one another reducing the size of spaces between adjacent slots 210. As the curve of the body lumen approaches a minimum radius of curvature of the sheath 200, the spaces between the slots 210 on the radially inner side of the curve close altogether. After the guide 200 has been inserted to the body lumen to a desired depth, the space 225 between the inner and outer tubes 220, 230, respectively, can be pressurized by supplying a fluid pressure P to the inflation port (not shown) as those skilled in the art will understand. As shown in FIG. 12, upon pressurization of the annular space 225, the inner tube 220 is adapted to be forced into the spaces the slots 210 facing the radially outer part of the curve occupying these spaces and thereby resisting changes to the shape of the elongated introducer 201 by preventing adjacent slots 210 from being moved toward or away from one another. As the outer tube 230 is less compliant than the inner tube 220, the bulk of the change in volume of the space 225 is directed toward movement of the inner tube 220 radially inward against the slotted member 201.

After the guide sheath 200 has been inserted to a desired position in the body lumen and stiffened by pressurizing the space 225, an endoscope or other instrument may be inserted through the lumen 250. After completion of a procedure, the space 225 may be deflated by opening the inflation port (not shown) or using any other known techniques such as, for example, the application of a vacuum, as those skilled in the art will understand, to return the sheath 200 to its more flexible state to facilitate removal or movement of the sheath 200 to a new location within the body lumen. The space 225 may then be re-stiffened, returned to its flexible state and moved to various locations within the body lumen as desired by repeating the above steps as often as desired. The sheath 200 is useful in procedures involving smaller body lumens as it is often necessary to inflate such lumens prior to insertion of an endoscope or instrument guide which function will be performed by the sheath 200.

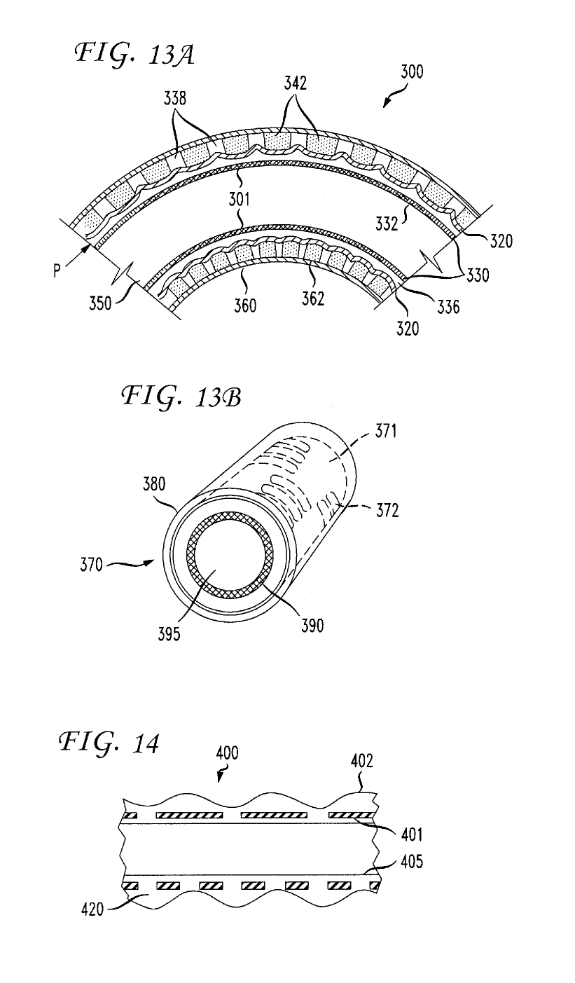

An alternate embodiment of the present invention, as shown in FIG. 13A, includes a guide sheath 300 comprising a slotted member 301 substantially similar to the slotted member 201 of the previous embodiment housed in a plurality of tubing layers, similar to those of FIGS. 11 and 12 except that the outer and inner tubes 320, 330, respectively, are positioned radially inside the slotted member 301. Thus, a radially inner surface 332 of the inner tube 330 forms a surface of a lumen 350 of the sheath 300 and a proximal inflation port (not shown) fluidly coupled to an annular space 336 between the inner and outer tubes 330, 320, respectively. A smooth outer surface 362 of the sheath 300 can be formed by a thin tube 360 formed, for example, of a compliant polymer material which surrounds the slotted member 301 to minimize trauma to the lumenal tissue and facilitate the insertion of the sheath 300 therethrough. As with the previously described embodiment, a distal end the tube 360 may be joined to the distal ends of the inner and outer tubes 330, 320, respectively, to form a substantially smooth atraumatic tip.

The outer tube 320 which can be located immediately radially inward from the slotted member 301, can be sealed to the inner tube 330 in the same manner described above for the tubes 220, 230 except that, in this embodiment, the outer tube 320 can be made more compliant than the inner tube 330 so that, when the space 336 is pressurized, the outer tube 320 is displaced radially outward into the spaces 338 between the slots 342 rigidizing the sheath 300 in the same manner described above in regard to the sheath 200.

FIG. 13B illustrates an alternate embodiment of the present invention, in which a guide sheath 370 comprises an elongated tube 371 with multiple slots 372 formed therein. As noted with regard to FIGS. 11-12, the slots 372 can enable the conduit 370 to conform to the curvature of body lumens into which it is inserted. An inner diameter of the elongated tube 371, which may be comprised of a flexible metal or plastic tube 390 may include, for example, an inner surface of braided material to form a surface of a lumen 395 extending through the sheath 370. Those skilled in the art will understand that the tube 390 can minimize trauma to endoscopes or other devices inserted through the lumen 395. Similarly, the outer diameter of the elongated tube 371 may be encased in a sleeve 380 comprised of a semi-rigid plastic material.

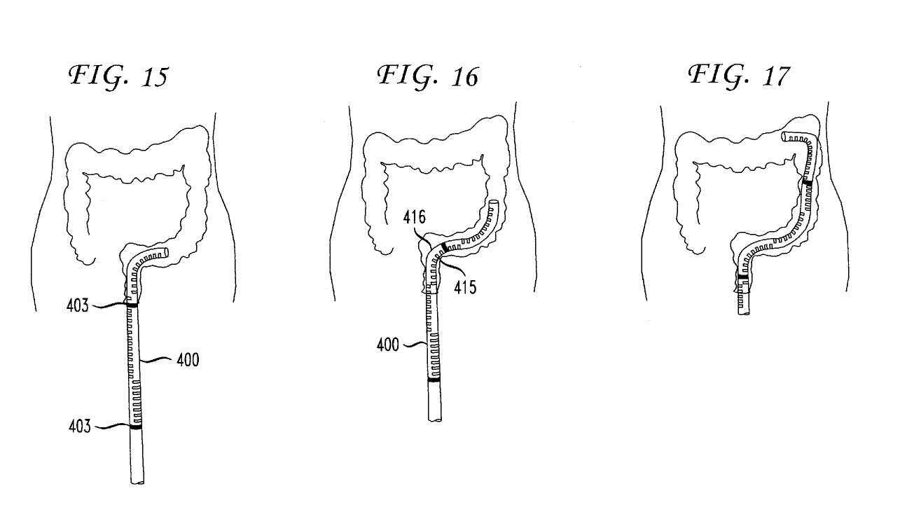

FIGS. 14-17 detail yet another embodiment of the present invention wherein a guide conduit 400 may be a slotted tube 401 with the arrangement of slots 420 in a slotted tube 401 altered in different areas of the conduit 400 to vary the stiffness and/or a preferred direction of bending of the conduit 400. For example, more closely spaced slots 420 placed on one side of the conduit 400 will make the conduit 400 more flexible on this side facilitating bending toward a direction which makes this surface an inner diameter of the conduit 400. As would be understood by those skilled in the art, the positioning of slots 420 may be specific to the type of procedure being performed and/or to the anatomy through which the conduit 400 is to be inserted. Specifically, the areas of increased flexibility may be positioned at locations corresponding to bends in the anatomy through which the conduit 400 is to be inserted with markings on the exterior of the conduit 400 indicating to a user a desired orientation of the sheath to facilitate navigation of the various anatomic bends. Furthermore, similar to embodiments described above outer tubing 402 and inner tubing 405 may be placed around outer and inner surfaces of the conduit 400 to prevent trauma to the lumen and endoscope, respectively.

As indicated above, the density of slots 420 along an inside 415 of a curvature may be increased relative to that of the outside 416 of the curvature to enhance bending in this direction. FIGS. 15-17 demonstrate a method by which the conduit 400 may be inserted into the lumen. Once the conduit 400 has passed through a first curvature in the lumen, as shown in FIG. 15, the conduit 400 can be rotated by 180 degrees to enable the retention of the aforementioned curvature when passing the conduit 400 through further curvatures. Position markers 403 placed along a length of the conduit 400 indicate to a diagnostician when a curvature has been passed and denote the rotational orientation of the conduit 400. Those skilled in the art will understand that the placement of areas of increased flexibility and the corresponding markers on the conduit 400 may vary according to the specific procedure being performed and the anatomy through which the conduit is to be inserted. Alternatively, the position markers 403 may be adapted to be visible under endoscopic imaging techniques known in the art (e.g., fluoroscopy, magnetic resonance imaging (“MRI”), etc.). Furthermore, the position markers 403 may also be employed in devices and methods for non-invasive medical procedures, as those skilled in the art will understand.

Once a second curvature has been passed, as shown in FIG. 16, the conduit 400 may be rotated again to conform to a next anatomic curve before inserting the conduit 400 further. Those skilled in the art will understand that the aforementioned embodiments may be altered in a number of ways in order to accommodate lumens of various sizes and curvatures.

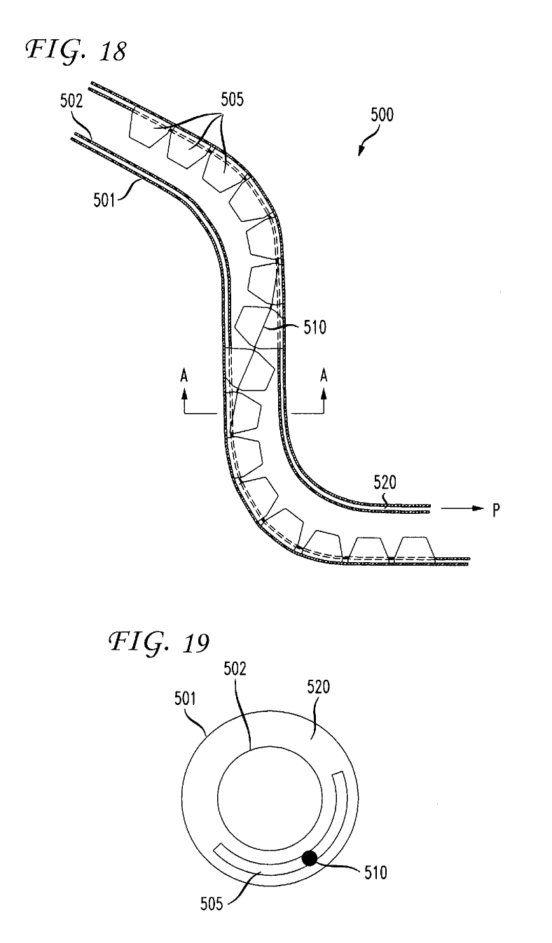

FIG. 18 illustrates a partial side view of yet another embodiment of the present invention. The guide sheath 500 of FIG. 18 is comprised of two coaxial tubes 501 and 502 with an annular space 520 between the two coaxial tubes 501 and 502 filled with a fluid. Submersed in the fluid are a series of curved plates 505 joined side to side along their central axes by a spine 510. Those skilled in the art will understand that the curved plates 505 may hold any suitable shape (i.e., oval, rectangular, etc.) and may be composed of any suitable elastomeric deformable material so that they may easily conform to the shape and curvature of a lumen into which the sheath 500 is to be inserted. The curved plates 505 may be connected loosely to the spine 510 to allow for ease of movement of each of the individual semi-circular plates 505 relative to one another.

Once the guide sheath 500 is in place in the lumen, a negative pressure P can be applied to a proximal end of the coaxial tubes 501 and 502 to remove the fluid from the annular space 520 tightening the coaxial tubes 501 and 502 and fixing the orientation of the plates 505 relative to one another. Consequently, the guide sheath 500 is adapted to stiffen increasing its resistance to changes in its shape in the lumen as it is subjected to forces (e.g., by an instrument inserted therethrough) and resisting imparting any forces to which it is subjected to the lumen within which it resides.

When it is desired to reposition the sheath 500 (e.g., after treatment has been completed), the annular space may again be filled with fluid to free the location of the spine 510 and the curved plates 505 for movement relative to one another enhancing the flexibility of the sheath 500 and facilitating the movement of the sheath 500 through the lumen.

Those skilled in the art will understand that the embodiment of the present invention may be modified in a number of manners without deviating from the scope of the invention. Instead of employing two coaxial tubes 501 and 502, any number of additional coaxial tubes may be used, with an increasing number of coaxial tubes enhancing the ability to fine tune a degree of stiffness imparted to the sheath 500 by selectively depressurizing certain of the annular spaces created while leaving others filled with fluid so that the performance characteristics of the device may be customized to differing needs at different phases of a procedure or simply by enhancing the overall stiffness by depressurizing all of the annular spaces. For example, a third middle coaxial tube may be added creating an additional annular space. Once the guide sheath 500 is placed in the desired position, the annular space between the middle layer and the inner coaxial tube 502 may be pressurized via the injection of a liquid or gaseous solution so that the pressurization forces the middle coaxial tube layer against the spine 510 and semi-circular plates 505 located between the middle coaxial tube and outer coaxial tube 501. This pressure exertion may help to fix the position of the spine 510 and semi-circular plates 505 in the guide sheath 500.

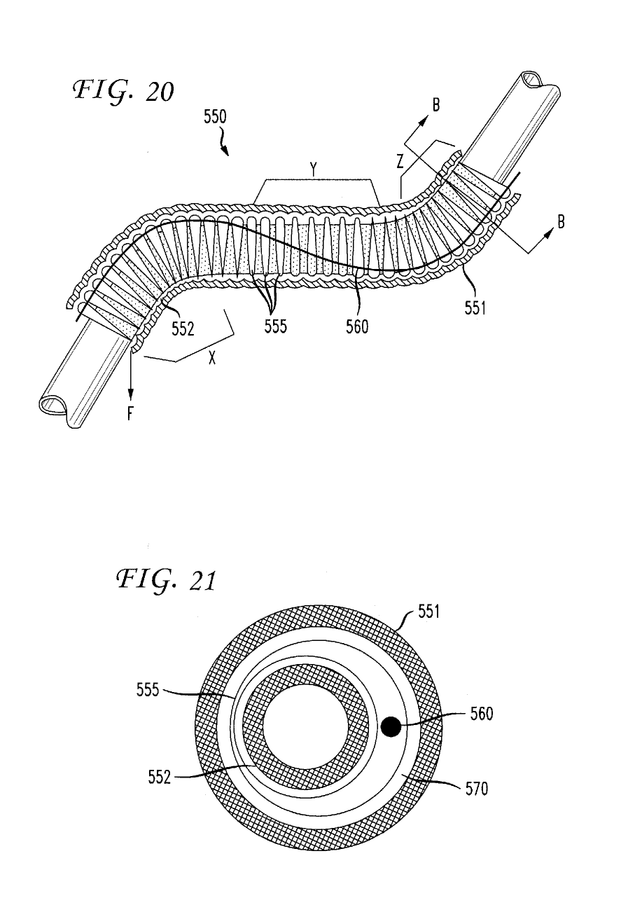

As shown in FIGS. 20 and 21, an alternate embodiment of the present invention may employ the use of torus or donut-shaped members 555 in place of the curved plates 505 of FIGS. 14 and 15. Whereas each of the curved plates 505 may encompass only a portion of the circumferential annular space 520, the donut-shaped members 555 may extend around the entire circumference of the annular space 570 which is sized to accommodate the donut-shaped members 555 on one side with a small clearance to allow for movement so that a portion of each the donut-shaped member 555 moves to a side of the annular space 570 located on an outer side of a radius of a curvature, as shown in FIG. 20. The donut-shaped members 555 of the guide sheath 550 may be connected to each other by a spine 560 extending longitudinally through the sheath 550.

As can be seen in FIG. 20 and 21, each of the donut-shaped members 555 can be shaped to produce a desired impact on the bending of the guide sheath 550. Specifically, first ends of the donut-shaped members 555 in areas X and Z can be sized to have a larger width than narrowed second ends to facilitate bending of the sheath 550 around radii on a side of the sheath 550 facing the second ends of these donut-shaped members 555 while the wider first ends of the donut-shaped members 555 can be arranged along an outer radius of such a curve. The donut-shaped members 555 of a central portion Y between the portions X and Z may be substantially symmetrical with respect to the axis of the sheath 550 or may gradually transition from the shape of the donut-shaped members 555 of portion X through a symmetrical center to gradually transition to the shape or the donut-shaped members 555 of portion Z.

FIG. 21 illustrates a cutout view of the guide sheath 550 along line B-B, wherein donut-shaped member 555 rest in the annular space 570 between the two coaxial tubes 551 and 552. As described earlier with respect to FIGS. 18 and 19, the application of a vacuum force F to remove a solution from the annular space is adapted to constrict the coaxial tubes 551 and 552 against one another fixing the orientation of the donut-shaped members 555 relative to one another.

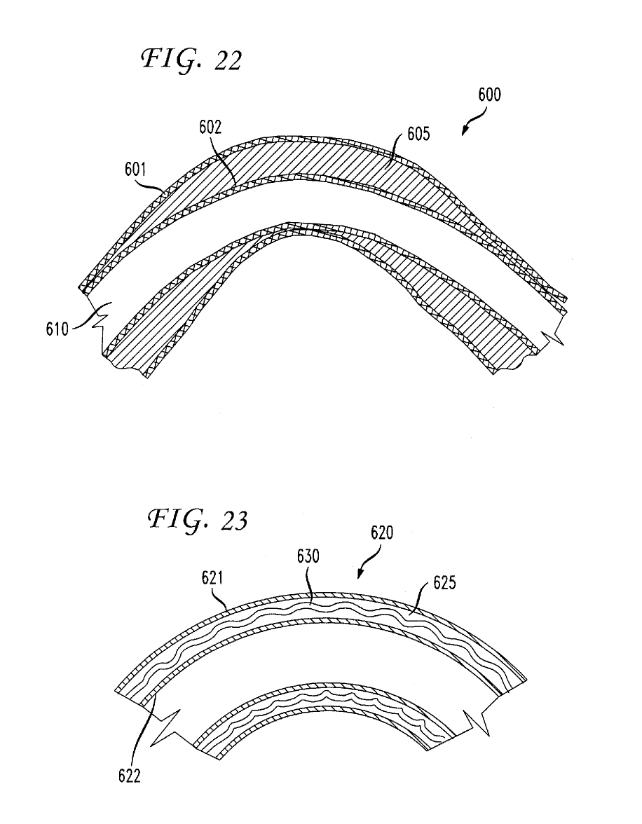

FIG. 22 details yet another embodiment of the present invention, wherein a guide sheath 600 includes coaxial tubes 601 and 602 with an annular space 605 situated therebetween. The coaxial tubes 601 and 602 may be composed of a semi-rigid material that providing sufficient stiffness for ease of insertion while remaining elastic enough to conform to the tortuous path of the body lumens into which it is to be inserted, as those skilled in the art will understand. The two coaxial tubes 601 and 602 can be joined to one another at a distal end to seal the annular space 605 while the proximal end of the annular space 605 may be accessible via an opening (e.g., a valve or other port) (not shown) so that materials may be provided to or removed from the annular space 605 as desired.

For example, the annular space 605 may be filled with an expandable foam filler material. Specifically, the foam filler may be formed with absorbent qualities, enabling the expansion thereof as would be understood by those skilled in the art. The sheath 600 may be preferably inserted through a body lumen to a desired position before the annular space 605 is filled with any material to provide maximum flexibility during insertion. After the sheath 600 has been inserted to the desired position, a foam-generating liquid solution can be injected into the opening at the proximal end of the sheath 600. As the liquid solution generates foam it expands inflating the annular space filling spaces between the tubes 601 and 602 created by curves in the guide sheath 600, stiffening the guide sheath 600 around the lumen 610 to facilitate passage of an endoscope therethrough. After the procedure necessitating the endoscope has been completed, the foam can be removed from the guide sheath 600 (e.g., after adding an agent to re-liquify the foam), relieving pressure in the annular space 605 and facilitating removal of the guide sheath 600 from the body lumen.

As illustrated in FIG. 23, a guide sheath 620 according to a further embodiment of the invention is substantially similar to the guide sheath 600 of FIG. 22, with a balloon sleeve 630 received in an annular space 625 formed by coaxial tubes 621 and 622. The balloon sleeve 630 may be sized to fit snugly in the annular space 625 when pressurized. Accordingly, the balloon sleeve 630 may be formed of a flexible, elastic material, so that it may easily conform to the shape of the sheath 620 as it curves through a body lumen. The balloon sleeve 630 can be sealed at a distal end thereof and includes an opening (e.g., a valve or other port) (not shown) accessible at a proximal end of the sheath 620 for supplying or withdrawing an inflation fluid to pressurize or depressurize the balloon sleeve 630. The guide sheath 620 is preferably inserted to a desired position within a body lumen while the balloon sleeve 630 is de-pressurized to maximize the flexibility of the sheath 620 and, after the desired position has been reached, inflation fluid can be supplied to the opening to pressurize the balloon sleeve 630, stiffening the guide sheath 620 and assisting it in maintaining its shape in the body lumen as devices are moved therethrough. When it is desired to remove the sheath 620 from the body lumen, the opening can be unsealed to deflate the balloon sleeve 630 returning the flexibility to the sheath 620.

Optionally, a distal portion of the outer coaxial tube 621 may be formed of an elastic material with a portion of the balloon sleeve 630 adjacent thereto, configured to expand to a greater diameter than other portions thereof. Thus, when the balloon sleeve 630 is inflated, the distal end of the balloon sleeve 630 may force the distal portion of the outer coaxial tube 621 radially outward to engage tissue of the lumen wall anchoring the sheath 630 in place. Those skilled in the art will understand that a separate anchoring balloon (not shown) may be formed on an outer surface of the outer coaxial tube 621 connected to an inflation lumen extending to a proximal end of the sheath 630 for supply and withdrawal of inflation fluid and that separate anchoring balloons and/or arrangements for the expansion of selected portions of the outer tube 621 to engage the body lumen may be formed at any locations along the length of the sheath 630.

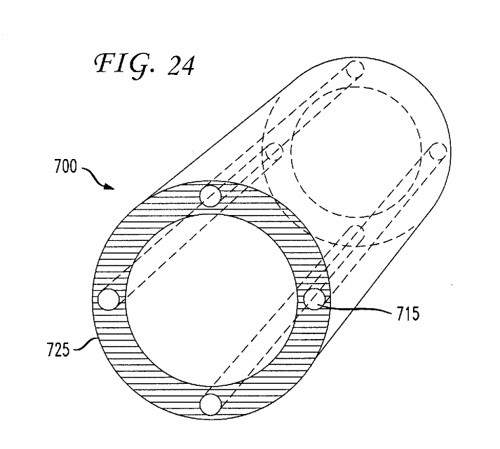

FIG. 24 illustrates a guide sheath 700 according to an embodiment of the present invention including an arrangement for hydraulic or pneumatic deflection comprising a plurality of longitudinal tubes 715 extending within an outer wall 725 thereof. Although FIG. 24 illustrates a sheath 700 including four longitudinal tubes 715, those skilled in the art will understand that any number of tubes 715 may be used to allow more precise control of the angle of deflection while less tubes 715 may be preferable to reduce the thickness of the sheath 700. The longitudinal tubes 715 can be sealed distal ends thereof and are selectively coupleable to a source of pressurized fluid at proximal ends thereof (e.g., via a valve or port) to allow for the pressurization and depressurization of these tubes 715 in combinations selected to achieve a desired deflection of the sheath 700. As would be understood by those skilled in the art, the pressurized fluid may be withdrawn from all of the tubes 715 before removal of the sheath 700 from the body to enhance the flexibility thereof.

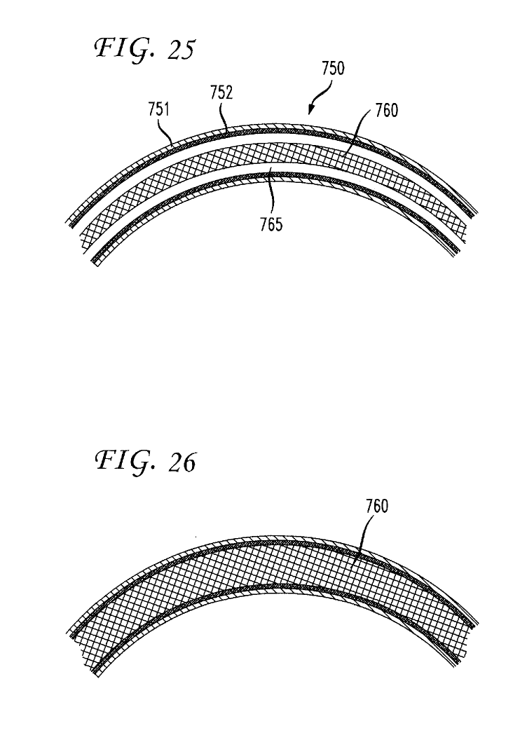

As shown in FIGS. 25 and 26, a sheath 750 according to yet another embodiment of the present invention comprises a guide sheath wall 751 defining a lumen 765 and covered internally by an elastomeric lining 752. The inner diameter of the guide sheath 750 may be composed of a high friction soft material such as silicon. Those skilled in the art will understand that the outer diameter of the guide sheath 750 may be composed of a material having rigidity and elasticity substantially similar to that of the outer layers of the previously described embodiments so that the guide sheath 750 may be inserted into body lumens in a flexible state and subsequently rigidized as described below to more effectively absorb forces applied thereto by instruments inserted through the sheath 750, minimizing the transfer of these forces to body tissues.

The guide sheath 750 is inserted to a desired position in a relatively flexible state and, after it has reached the desired position, an expandable metal tube 760 can be passed through the lumen 765 in a reduced diameter configuration to the distal end of the sheath 750. The tube 760, which may be an intertwined braid or coil of material possessing spring-like qualities (e.g., metal, polymers, etc.) is held in the reduced diameter configuration, for example, under tension, as shown in FIG. 26. For example, the tube 760 may be formed as a coil which may be twisted into a reduced profile insertion configuration and which may, when in the target location, be freed to unwind into an expanded deployed configuration, as shown in FIG. 26. As would be understood by those skilled in the art, any known means may be used to hold the tube 760 in tension including, for example, a clip or other locking mechanism coupled to a proximal end thereof. After the tube 760 has been properly positioned in the lumen 765, the tension may be released (e.g., by removing the locking mechanism) allowing the tube 760 to expand and engage the elastomeric lining 752, stiffening the sheath 750. As would be understood by those skilled in the art, the tube 760 may be encased in an elastic liner in order to prevent trauma to an endoscope or other device to be inserted through the lumen 765. When it is desired to remove the sheath 750 from the body lumen, the tube 760 can be returned to the reduced diameter configuration (e.g., by re-coupling the locking mechanism to the proximal end) and withdrawn from the lumen 765 to return flexibility to the sheath 750. The flexible sheath 750 can then withdrawn from the body lumen.

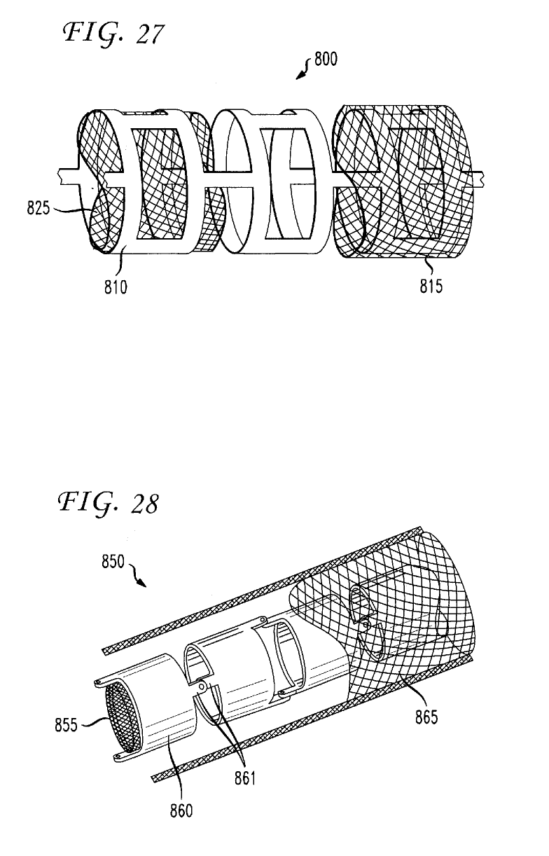

As shown in FIG. 27, a guide sheath 800 according to another exemplary embodiment of the present invention comprises an outer sleeve 815 and an inner braid 825 similar to the outer sleeve 380 and the inner braid 390 employed in the embodiment of FIG. 13B except that the tube 810 of this embodiment comprises a thin metal material from which portions have been removed (e.g., via photo-lithographic etching), as those skilled in the art will understand. For example, a pattern can be first etched on a flat sheet of thin metal. After completion of the etching, the sheet can be rolled and welded to form the substantially cylindrical tube 810. Those skilled in the art will understand that the etched tube 810 enhances the flexibility of the sheath 800 allowing the sheath 800 to more easily conform to curvatures of the body lumens into which it is inserted.

As shown in FIG. 28, a guide sheath 850 according to another exemplary embodiment of the invention comprises a series of segments 860 longitudinally attached to one another with a distal end of each segment 860 attached to a proximal end of an adjacent segment 860 via protruding members 861 extending from the distal and proximal ends of the segments 860. The attachment of adjacent segments 860 may be any means known in the art (e.g., latching, etc.) allowing for rotational movement of segments 860 relative to adjacent segments 860 about axes of rotation substantially perpendicular to a longitudinal axis of the sheath 850 allowing the sheath 850 to bend and conform to the curvature of a body lumen into which it is inserted. Adjacent segments 860 may be connected to one another with a substantial friction fit to aid in the assumption of a desired configuration, as those skilled in the art will understand. The sheath 850 can include an outer sleeve 865 and an inner braid 855 similar to those of FIG. 27 to minimize trauma to both the body lumen and an endoscope or other device to be inserted through the guide sheath 850.

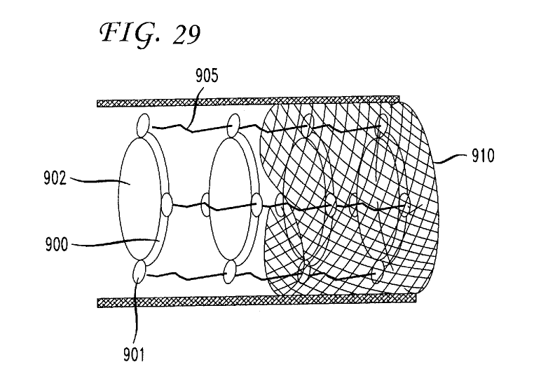

As shown in FIG. 29, a guide sheath 900 according to a further embodiment of the present invention includes smaller metal or plastic links 905 in place of the segments 860. These links 905 can be connected longitudinally via a series of wires 910 (e.g., copper wires) running the length of a conduit 900 through a lumen 902 extending therethrough. The links 905 may have attachment points 901 for attachment of the wire 910, wherein the attachment may be done using techniques known in the art (i.e., glue, soldering, etc.). The conduit 900 may include an outer sleeve 920 and an inner sleeve (not shown) as described in regard to the previous embodiments to prevent trauma to devices inserted through the lumen 902 and to lumenal tissue.

It is further noted that any combination of the above listed embodiments and components thereof is contemplated. For example, the guide sheath 800 of FIGS. 27 and 28 may further comprise inflatable elements to enhance positioning in the body. Furthermore, any of the elements in any embodiment disclosed herein may be grouped or aligned in a discontinuous manner. Specifically, it is noted that there is no requirement for successive elements in the disclosed embodiments to be connected to one another. Rather, embodiments of the present invention are directed to the placement of successive elements at key points along the sheath such as, for example, points which, when deployed to a target location, are likely to be subjected to increased pressure, or which are likely to lie along a curve of the path along which a device is to be inserted, etc.

The present invention has been described with reference to specific exemplary embodiments. Those skilled in the art will understand that changes may be made in details, particularly in matters of shape, size, material and arrangement of parts. Accordingly, various modifications, combinations and changes may be made to the embodiments. For example, each embodiment of the guide conduit may provide visual guidance with the employment of an Imaging Sensor (e.g., a CMOS sensor) located at the distal tip of the guide sheath with illumination provided by, for example, LEDs or small plastic fibers running inside the wall of the guide sheath. Another possible modification may comprise the addition of tracking means to the guide sheath to track the progress of the sheath through the anatomy and to aid in placement of the sheath. Such means may include magnetic proximity sensors, radiopaque markers, infrared (thermal) emitters and electronic tracking components (active and passive) as would be understood by those skilled in the art. Yet another possible modification of the present invention may include the addition of the capability for Narrow Band Imaging (NBI) for the identification of abnormal tissue, as those skilled in the art will understand.

In another alternate embodiment of the present invention, longitudinal sections of the guide tube such as, for example, a distal section, may be adapted to exhibit a greater degree of flexibility than other portions. The specifications and drawings are, therefore, to be regarded in an illustrative rather than a restrictive sense.

What is claimed is:

1. A guide conduit for facilitating insertion of instruments into the body, the conduit defining a lumen extending from an opening at a proximal end of the conduit to a distal opening at the distal end of the conduit, the conduit comprising:
an outer layer forming a substantially smooth outer surface; and
a deformable layer radially within the outer layer, the deformable inner layer constructed to maintain its shape when subjected to a force below a predetermined threshold level and to assume a new shape when subjected to a second force greater than the threshold level, wherein the threshold level is selected to be greater than a range of forces to which the conduit will be subjected by instruments inserted therethrough.

2. The guide conduit according to claim 1, wherein the deformable layer comprises a plurality of elements rotatably coupled to one another and extending along an axis of the conduit, each of the elements extending around a portion of a circumference of the lumen with a frictional force preventing relative rotation between adjacent ones of the elements when the conduit is subjected to a bending force less than the threshold level.

3. The guide conduit according to claim 2, wherein each of the elements is coupled to an adjacent one of the elements for rotation about an axis substantially perpendicular to an axis of the conduit and to prevent rotation relative thereof about an axis parallel to the axis of the conduit.

4. The guide conduit according to claim 2, further comprising a wire coupled to the elements so that tension applied to the wire draws the elements against one another increasing a stiffness of the conduit.

5. The guide conduit according to claim 1, wherein the inner lumen of the conduit comprises a lubricious material.

6. The guide conduit according to claim 2, wherein the elements are shaped differently at selected bending locations along the conduit and wherein the elements forming the bending locations include narrow ends facing a desired bending radius.

7. The guide conduit according to claim 1, wherein the deformable layer includes a thin layer of metal including voids sized to achieve a desired threshold level, the voids being positioned to facilitate bending in desired directions.

8. The guide conduit according to claim 1, wherein the deformable layer comprises a slotted portion, slots of the slotted portion being distributed asymmetrically to form first areas showing enhanced flexibility relative to second areas.

9. The guide conduit according to claim 1, further comprising an expandable coil layer received within the deformable inner layer and extending along an axis of the conduit.

10. The guide conduit according to claim 1, further comprising an expandable coil sized to receive the guide conduit therewithin.

11. A guide conduit for facilitating insertion of instruments into the body, the conduit defining a lumen extending from an opening at a proximal end of the conduit to a distal opening at a distal end of the conduit, the conduit comprising:
an outer layer forming a substantially smooth outer surface;
an inner layer separated from the outer layer to form an annular space therebetween; and
a fluid access port at a proximal end of the conduit.

12. The guide conduit according to claim 11, further including a balloon member received within the annular space in fluid communication with the port, wherein the outer layer includes an inflatable portion at a distal end thereof so that, upon inflation of the balloon member, an elastic portion of the outer layer expands to engage tissue of a body lumen within which the conduit is located, the balloon member further including an increased diameter portion at a distal end thereof, the increased diameter portion corresponding in position to the elastic portion of the outer layer.

13. The guide conduit according to claim 11, including a slotted layer within the annular space and a balloon member received within the annular space in fluid communication with the port, the balloon being located between the slotted layer and the outer layer of the conduit, a surface of the balloon member facing the outer layer being less inflatable than a surface of the balloon member facing the slotted layer, slots of the slotted layer being distributed asymmetrically to form bending areas of enhanced flexibility

14. The guide conduit according to claim 11, further including a balloon member received within the annular space in fluid communication with the port, a slotted layer being located between the balloon member and the outer layer of the conduit, wherein a surface of the balloon member facing the slotted layer is less inflatable than a surface of the balloon member facing away from the slotted layer, slots of the slotted layer being distributed asymmetrically to form bending areas of enhanced flexibility.

15. The guide conduit according to claim 11, further comprising a deformable layer received within the annular space, the deformable layer being formed as a series of partially circumferential curved plates rotatably coupled to one another and extending along an axis of the conduit, the deformable layer comprising a spine extending along an axis of the conduit

16. The guide conduit according to claim 15, wherein the deformable layer is formed as a series of torus shaped members rotatably coupled to one another and extending along an axis of the conduit, first ends of the torus shaped members comprising a larger width than narrowed second ends to facilitate bending toward the second ends.

17. The guide conduit according to claim 11, wherein the annular space comprises a fluid lumen extending along an axis of the conduit.

18. A method of inserting an endoscope into a body of a patient, comprising:
advancing the endoscope into a body by a first predetermined distance;
advancing a substantially tubular hollow guide over the endoscope and into the body by a second predetermined distance greater than the first predetermined distance; and
sequentially advancing the endoscope and the substantially tubular hollow guide distally into the body until a target location is reached.

19. The method of claim 18, further comprising:
rotating the substantially tubular hollow guide when passing a first curvature in the body, wherein the location of the first curvature is indicated by a marking on the substantially tubular hollow guide.

20. A method of inserting an endoscope into a body of a patient, comprising:
advancing a guide conduit into a body, the guide conduit defining a lumen extending from an opening at a proximal end of the conduit to a distal opening at a distal end of the conduit, the conduit comprising an outer layer forming a substantially smooth outer surface and an inner layer separated from the outer layer to form an annular space;
infusing a fluid into a fluid access port at a proximal end of the conduit, the fluid access port opening into the annular space and causing the conduit to rigidize; and
withdrawing the fluid from the fluid access port to increase flexibility of the conduit.
